(19) United States
(12) Patent Application Publication
(10) Pub. No.: US 2004/0067904 A1
(43) Pub. Date: Apr. 8, 2004

(54) Gene therapy for prostate cancer: sensibilization of cells to dna damaging drugs and radiation

(76) Inventors: Viatcheslav A. Soldatenkov, Washington, DC (US); Mira Jung, Washington, DC (US); Anatoly Dritschilo, Washington, DC (US)

Correspondence Address: PILLSBURY WINTHROP, LLP, P.O. BOX 10500, MCLEAN, VA, 22102, US

(21) Appl. No.: 10/450,411

(22) PCT Filed: Dec. 13, 2001
(86) PCT No.: PCT/US01/47948

(51) Int. Cl. A61K 48/00; C07H 21/04

(52) U.S. Cl. 514/44; 536/23.5; 435/320.1

(57) ABSTRACT

The present invention is directed to a novel therapeutic method for treating prostate cancer. The method employs the tissue-specific PSA promoter/enhancer as well as the unique properties of the DNA binding domain (dbd) of poly (ADP-ribose) polymerase (PARP) as a potent inhibitor of DNA damage repair and as a molecular sensitizer to genotoxic stresses. The sustained presence of the PARP-DBD in prostate tumor tissue induces enhanced tumor cell killing in response to DNA damaging treatments. The invention may be used as a biotherapeutic approach in the treatment of prostate cancers, which fail local-regional therapy, without significant risk of normal tissue damage.

### BACKGROUND OF THE INVENTION

1. Related Applications

The present application claims priority to U.S. Provisional Application Serial No. 60/254,924, filed Dec. 13, 2000, the contents of which are hereby incorporated by reference in their entirety, 2. Summary of the Related Art

Normal tissue homeostasis is achieved by an intricate balance between the rate of cell proliferation and the rate of cell death. Disruption of this balance is thought to be a major event in the development of cancer. The inhibition of apoptosis, or programmed cell death, has been linked to this disruptive event. The effects of cancer are catastrophic, causing over half a million deaths per year in the United States alone.

Several studies suggest that treatment of tumors with DNA damaging agents results in up-regulated DNA damage repair mechanisms, which could account for increased resistance to DNA damaging therapy. In normal cells, DNA damage results in cell cycle arrest and induction of DNA repair mechanisms, so as to prevent the transfer of damaged DNA to the next generation of cells. Cells that sustain high levels of DNA damage, such as tumor cells that exhibit high levels of karyotypic instability, or cells that are treated with DNA damaging agents, are induced to undergo apoptosis.

It is, therefore, a goal of the present invention to provide improved methods for the treatment of cancer. More particularly, it is a goal to provide methods for overcoming or limiting the therapy-inhibiting effects of DNA repair in cancer cells.

Prostate cancer is the most common malignancy in men of all ages and is becoming a public health problem in the United States. After lung cancer, prostate cancer is the second most fatal cancer among American males with approximately 40,000 deaths annually (Wingo et al., J. Clin. 45: 8-30 (1995)). While more than 80% of the tumors initially respond to androgen ablation, metastatic prostate cancer inevitably progresses to an androgen-independent state (Isaaks et al., D. Development of androgen resistance in prostatic cancer. In: G. Murphy, S. Khoury, I. Kuss, and C. Chatelain (eds.), Prostate cancer, Part A: Research, Endocrine Treatment, and Histopathology, pp. 21-3 I. New York: Alan R. Liss, Inc. (1987)). Once this occurs, control of the disease is difficult since hormonally-independent tumors become resistant to further hormonal manipulations as well as chemotherapy and radiotherapy (Mahler et al., Cancer, 70: 329-34 (1992); Raghavan, Semin. Oncol., 15: 371-89 (1988); Schellhammer, Radiation therapy for localized prostate cancer: what has been and still remains to be learned. In: T. Stamey (ed.), Monographs in Urology, 15 (15). Stanford, Calif.: Medical Directions Publishing Co., Inc. (1994)). Radiation therapy has been proven to be a relatively effective treatment for localized prostate cancer, resulting in 15-year rates of disease-free survival of 45 to 85% of patients (Catalona, N. Engl. J. Med., 331: 996-1004 (1994). However, tumor size, microenvironmental factors such as hypoxia, and the presence of radio-resistant tumor cells limit radio-curability (Schellhammer, P. F. Radiation therapy for localized prostate cancer: what has been and still remains to be learned. In: T. Stamey (ed.) Monographs in Urology, 15 (15). Stanford, Calif.: Medical Directions Publishing Co., Inc. (1994)).

The role of Programmed Cell Death in Prostate Cancer.

Recent studies have shown that cancer is the result of multiple genetic events, which cause the loss of growth control or inhibition of appropriate cell death. The majority of prostatic cancer cells in the metastatic site are not actively proliferating (Berges et al., Clin. Cancer Res., 1: 473-480 (1995)) and are thus resistant to standard therapies (Mahler et al., Cancer, 70: 329-334 (1992); Raghavan, Semin. Oncol., 15: 371-389 (1988); Catalona, N. Engl. J Med., 331: 996-1004 (1994)). The activation of programmed cell death pathways in response to ionizing radiation or chemotherapy has been recognized as a primary determinant of the radio-sensitivity of various tumor cells (Stephens et al., Radiat. Res., 135: 75-80 (1993)). Human prostate carcinoma cells retain the ability to undergo programmed cell death upon stimulation by diverse agents including radiation (Kyprianou, World J. Urol., 13: 299-303 (1994); Rarnsamooj et al., Radiat. Oncol. Invest, 3:346-352 (1996); Prasad et al., Biochem. Biophys. Res. Communs, 249: 332-338 (1998)). Therefore, apoptosis has emerged as a main therapeutic target for the effective elimination of prostate cancer cells in response to radiation or chemotherapy (Kyprianou (1994); Fisher, Cell, 78: 593-542 (1994); McConkey et al. Cancer Res., 56: 5594-5599 (1996)). For these reasons increased attention is being paid to the combined use of chemotherapy or radiotherapy and molecularly targeted drugs that directly activate apoptotic pathways or indirectly activate apoptotic pathways via inhibiting DNA damage repair. Identification of radio-sensitizing biological compounds will enable the optimization of radiation therapy for the treatment of advanced prostate cancer by enhancing the therapeutic response to radiation or chemotherapy while circumventing the problem of systemic toxicity associated with the doses of chemotherapy or radiation currently used for radio-responsive lesions.

Poly (ADP-Ribose) Polymerase: the Role in DNA Damage Repair and Apoptosis.

Poly(ADP-ribose) polymerase (PARP) is an abundant nuclear protein that is associated with chromatin. The role of this enzyme is to catalyze the transfer of the ADP-ribose moiety from nicotinamide adenine dinucleotide (NAD⁺) to itself and other nuclear acceptor proteins to form ADP-ribose polymers.

PARP is a zinc finger-containing protein, which allows the enzyme to bind to either double- or single-strand DNA breaks without any apparent sequence preference. The catalytic activity of PARP is strictly dependent on the presence of strand breaks in DNA, and is modulated by the level of automodification (Cleaver, et al., Mutat. Res., 257:1-18 (1991); Satoh, et al., Nature (Lond.), 356:356358 (1992)). Cell culture systems have demonstrated that PARP is involved in numerous biological functions, all of which are associated with the breaking and rejoining of DNA strands (Berger et al., Biochemistry, 19: 289-293 (1980); Satoh et al., J. Biol. Chem., 268: 5480-5487 (1993); Smulson et al., Biochemistry, 33: 6186-6191 (1994); Stevnser et al., Nucleic Acids Res., 22: 4620-4624 (1995)). It has been observed that PARP is also involved in regulating nuclear functions and cell differentiation (reviewed in Soldatenkov and Smulson, Int. J. Cancer, 90:59-67 (2000)).

Mice lacking PARP as a result of gene disruption exhibit diverse phenotypes. Whereas animals of one strain show epidermal hypertrophy and obesity (Wang et al., Genes Dev., 9: 509-520 (1995)), those of another strain exhibit growth retardation, aberrant apoptosis, and increased sensitivity to DNA-damaging agents (De Murcia et al. Proc. Natl. Acad Sci. USA, 94: 7303-7307 (1997)). Eukaryotic cells expressing PARP antisense cDNA have a pronounced lag in initiation of DNA repair (Stevnser et al., supra; Ding et al., J. Biol. Chem., 267: 12804-12812 (1992)), which results in altered chromatin structure and reduced survival after exposure to DNA-damaging agents (Ding et al., Cancer Res., 54: 4627-4634 (1994)). These data indicate that PARP plays a pivotal role in DNA damage repair. It has been hypothesized that PARP cycles between an unmodified form, which blocks DNA strand ends, and a modified form, which is released from DNA, thereby allowing access of repair enzymes to the site of damage (Satoh et al., supra). The “PARP cycling” was recently demonstrated in an in vitro DNA repair system using deletion mutants of PARP (Smulson et al., supra).

It has been shown that limited proteolysis of PARP by the caspase family of cysteine proteases is an early event or perhaps a prerequisite for the execution of programmed cell death in a variety of cells (Kaufman et al., Cancer Res., 53: 3976-3985 (1993); Soldatenkov et al., Cancer Res., 55: 4240-4242 (1995); Soldatenkov et al., Int. J. Oncology, 9: 547-551 (1996)). The caspase-specific DEVD motif resides adjacent to the nuclear localization signal of PARP protein. Cleavage of PARP at this site (between Asp 216 and Gly 217) results in the separation of the two zinc finger DNA-binding motifs in the amino terminus of PARP from the automodification and catalytic domains located in the carboxyl terminus of the enzyme (Nicholson et al., Nature, 376: 37-43 (1995)). Consequently, this cleavage excludes the catalytic domain from being recruited to the sites of DNA fragmentation during apoptosis and presumably disables PARP from coordinating subsequent repair of genome maintenance events (Smulson et al., supra).

DNA-Binding Domain of the Poly(ADP-Ribose) Polymerase: a Potential Molecular Radio-Sensitizer.

The PARP-DBD fragment is a naturally generated product of limited proteolysis of PARP by caspases at the execution stage of programmed cell death (FIG. 1). It acts as a trans-dominant inhibitor of PARP activity by competing with intact PARP for DNA strand breaks (Kupper et al., J. Biol. Chem., 1990 265: 18721-18724 (1990); Schreiber et al., Proc. Natl Acad. Sci. USA, 92: 4753-4757 (1995); Kupper et al., Molec. Cell Biol., 3154-3163 (1995)). Preliminary data has shown that PARP-DBD irreversibly binds to broken DNA strands (Smulson et al., Cancer. Res., 58: 3495-3498 (1998)). These unique properties of the DNA-binding domain of PARP suggest its biological function—to block the lesions in DNA and make them inaccessible for DNA repair enzymes followed by disposal of the cells that sustain unrepaired DNA damage via apoptosis.

The biological function of DNA-binding domain of PARP (PARP-DBD) has been investigated by utilizing stable cell lines constitutively expressing DBD (Kupper et al., J. Biol. Chem., 265: 18721-18724(1990); Schreiber et al., Proc. Natl. Acad. Sci. USA, 92: 4753-4757 (1995); Kupper et al., Molec. Cell Biol., 15: 3154-3163 (1995)). Data obtained from these experiments indicate that DBD expression in mammalian cells (i) led to trans-dominant inhibition of PARP, (ii) had no effect on normal cell proliferation, and (iii) sensitized the cells to genotoxic agents such as N-methyl-N′-nitro-N-nitrosoguanidine (MNNG) and ionizing radiation.

The sensitization of DBD-expressing mammalian cells to ionizing radiation and DNA-damaging agents has been recently demonstrated (Kupper et al., J. Biol. Chem., 1990265: 18721-18724 (1990); Schreiber et al.) Proc. Natl Acad. Sci. USA, 92: 4753-4757 (1995); Kupper et al., Molec. Cell Biol., 3154-3163 (1995); Kupper et al., Cancer. Res., 56: 2715-2717 (1996)). Previous studies have shown that exposure of DBD-expressing cells to DNA damaging agents resulted in a marked reduction of cell survival, increased frequency of sister chromatid exchanges, inhibition of cell proliferation and apoptosis induction (Kupper et al., (1990) supra); (Schreiber et al., supra); Kupper et al., (1995) supra); Kupper et al., Cancer. Res., 56: 27152717 (1996)). DBD-induced apoptosis has to be independent of cell proliferation states since both nondividing neurons and rapidly proliferating cancer cells can not survive the massive accumulation of long-lived damage in genome (Blank et al., Int. J. Radiat. Biol., 71: 455-466 (1997)). This characteristic is in marked contrast to that of most conventional chemotherapeutic drugs, which induce cytotoxicity only in proliferating cells. This feature is a great advantage, especially for prostate cancer treatment, which grows very slowly in general and is highly invasive and metastatic (Berges et al., (1995) supra).

Thus, PARP-DBD functions to interrupt cell division of DNA-damaged cells and to eliminate these cells by initiating the cellular suicide program. Therefore, the DBD of PARP may have a potential for targeted sensitization of tumor cells to radiotherapy and chemotherapy.

Prostate Tissue-Specific Promoters in Targeting Gene Therapy.

In the past few years, several new approaches for treating advanced neoplasms have been proposed, including that of gene therapy (Hrouda et al., Gene Therapy, 3: 845-852 (1996)). Differential expression of the desired gene product in the target tissue is central to the concept of gene therapy. One such approach is to use tissue-specific promoters, such as the prostate-specific antigen promoter, to drive therapeutic genes (Hart, Semin. Oncol., 23: 154-158 (1996); Denmeade et al., Cancer J. Sci. Am., 4: 15-21 (1998); Gotch et al., J. Urology, 160: 220-229 (1998); Denmeade et al., Cancer Res., 58: 2537-2540 (1998)). Predominant expression of prostate-specific antigen in prostate and activation of its expression in most patients with prostate cancer renders the PSA promoter an ideal regulatory element for conferring prostate-specific transgene expression. Thus, the prostate-specific antigen (PSA) promoter would be a promising tool for prostate cancer-specific gene expression (Denmeade et al, Cancer J. Sci. Am., 4: 15-21 (1998); Gotoh et al., J. Urology, 160: 220-229 (1998); Demneade et al., Cancer Res., 58: 2537-2540 (1998)).

PSA is a Mᵣ 34,000 member of the human kallikrein gene family and is synthesized by normal, hyperplastic, and malignant prostatic epithelial (Clements, Endocrin. Rev., 10: 393-419 (1989)). Although low levels of PSA are detectable in the serum of men with normal prostates, PSA expression is increased in most patients with prostate cancer, regardless of tumor stage and hormone responsiveness. Clinically, PSA has been used as a serum tumor marker for the diagnosis and follow-up of prostate cancers (Murphy et al., Cancer, 83: 2259-2269 (1998); Sweat et al., Urology, 52: 637-640 (1998).

The promoter of the PSA gene has been cloned and its two functional domains have been identified: a proximal promoter and a distal promoter, which can also function as an enhancer (Riegman et al., Mol. Endocrinol., 5: 1921-1930 (1991); Pang et al., Human Gene Therapy, 6: 1417-1426 (1995); Schuur et al., J. Biol. Chem., 271: 7043-7051 (1996); Cleutjens et al., J. Biol. Chem., 271: 6379-6388 (1996); Pang et al., Cancer Res., 57: 495-499 (1997)). Three androgen-responsive elements have also been identified within the 5′ flanking region of the PSA gene (Schuur et al, supra; Cleutjens et al., supra).

Using LNCaP tumor xenografts in the nude mouse model it was demonstrated that the PSA promoter retained its tissue-specific properties in vivo (Schuur et al, supra). Furthermore, the PSA promoter fragment was able to mimic the prostate-specific and androgen-regulated expression of the PSA gene in transgenic mice (Wei et al., Proc. Natl Acad. Sci. USA, 94: 6369-6374 (1997)). Thus, the PSA promoter has demonstrated the features that are fundamental to the development of expression vectors for prostate-specific gene therapy: tissue specificity and androgen responsiveness.

### SUMMARY OF THE INVENTION

Tumor cell resistance to chemotherapeutic drugs and radiation represents a major problem in clinical oncology. Higher doses of drugs or ionizing radiation may improve the response rate in some malignancies, but these treatment methods also cause increased toxicity in the host. This is particularly true in cases of prostate cancer where the majority of prostate cancer cells are not actively proliferating and are thus resistant to standard cytotoxic therapies. Furthermore, these current methods of cancer therapy, such as radiation therapy and chemotherapy, are either not effective against human prostate cancer or are not specific for prostate carcinoma cells. Accordingly, there is a need in the art for novel methods of treating cancer, in particular, prostate cancer by molecularly targeting drugs that offer the potential of enhancing tumor cell responses to genotoxic treatments while minimizing side effects associated with toxicity.

To that end, the present inventors have discovered a method of treating prostate cancer comprising a combination therapy that utilizes tissue-specific and treatment-specific therapies for prostate cancer. Specifically, the present invention provides a novel method comprising a gene therapy delivery system with tissue-specific expression of a DNA repair inhibiting gene, DBD-PARP, combined with the administration of potent DNA-damaging agents.

Thus, it is an object of the invention to provide a novel recombinant DNA construct comprising the coding region of the DNA binding domain of PARP linked to the tissue-specific PSA promoter and enhancer.

It is another object of the invention to provide an expression vector comprising the coding region of the DNA binding domain of PARP linked to the tissue-specific PSA promoter and enhancer.

It is yet another object of the invention to provide a method for expressing the DNA-binding domain of poly(ADP-ribose) polymerase under the control of the prostate specific antigen promoter in prostate carcinoma cells.

It is another object of the invention to provide a method for treating cancer comprising sensitizing cancer cells to DNA-damaging therapies by administering to a host a therapeutically effective amount of a gene therapy composition comprising a construct of the invention and killing the targeted cancer cells by inducing DNA damage and apoptosis.

DNA-damaging agents or factors are defined herein as any chemical compound or treatment method that induces DNA damage when applied to a cell. Such agents and factors include radiation and waves that induce DNA damage, such as irradiation, X-rays, microwaves, electronic emissions, and the like. A variety of chemical compounds, also described as “Chemotherapeutic agents” function to induce DNA damage, all of which are included to be of use in the combined treatment methods disclosed herein. Chemotherapeutic agents contemplated to be of use, include alkylating agents (e.g. cisp-diamine dichloroplatinum (CDDP) or melphalan), agents that interfere with DNA replication, mitosis, and chromosomal segregation (e.g. etoposide (VP-16), camptothecin and adriamycin, also known as doxorubicin), radiomimetic agents (e.g. bleomycin). In certain embodiments, the use of γ-irradiation in combination with a PARP-DBD expression in prostate is particularly preferred as this compound.

In addition, to avoid the potential side effects that may occur due to expression of the PSA construct of the invention in tissues other than prostate, an agent that is not functionally active in the absence of massive DNA damage will be used to target tumor cells. Therefore, there will be no toxic effects outside the treated area.

Thus, it is another object of the invention to provide an agent such as the PARP-DBD, which is not functionally active in the absence of massive DNA damage, to prevent potential side effects due to the expression of the PSA construct of the invention in tissues other than prostate.

### BRIEF DESCRIPTION OF THE DRAWINGS

FIG. 1: Model for LIFE/DEATH regulatory functions of the PARP and the PARP-DBD in response to DNA damages. PARP acts as a positive element in DNA damage repair (a nick-protection mechanism of polymerase action) so that coordinated PARP-DNA interactions allow access for DNA lesions. In contrast, irreversible binding of the DBD of PARP to DNA results in fixation of DNA lesions followed by apoptosis induction to eliminate cells retaining substantial long-lived genomic damage. This model represents the conceptual framework for sensitization of prostate cancer to genotoxic treatments by expression of DNA-binding domain of PARP.

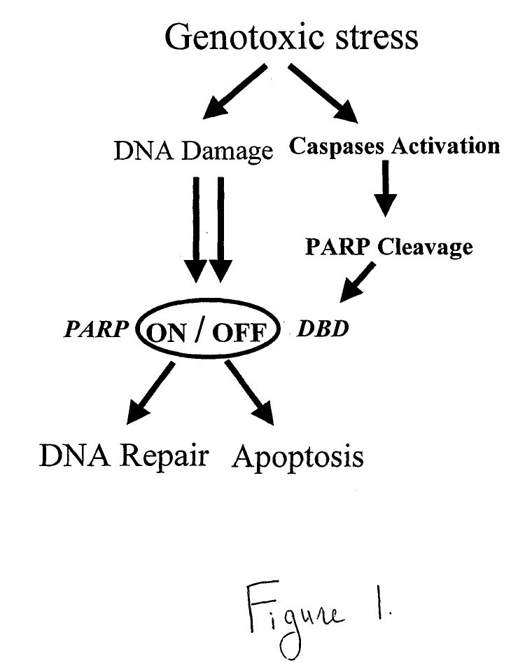

FIG. 2: Atomic force microscopy revealed that recombinant full length PARP bound plasmid DNA fragments and linked them into chainlike structures (A). Automodification of PARP in the presence of NAD⁺resulted in its dissociation from the DNA fragments, which, nevertheless, remained physically aligned (B). A recombinant 28-kDa fragment of PARP containing the DNA-binding domain but lacking the automodification domain irreversibly bound to and linked DNA fragments in the absence (C) or presence (D) of NAD⁺.

FIG. 3: A schematic representation of the recombinant constructs for constitutive (pCMV-DBD) and androgen-inducible (pPSA-DBD) expression of the human PARP-DBD in prostate cancer cells. PSA-E, 1.3 kb upstream PSA enhancer region; PSA-P, 0.6 kb minimal promoter of the human prostate-specific antigen (PSA) gene. Relevant restriction enzyme sites, zinc fingers (Zn) and androgen response elements (ARE) are indicated.

FIG. 4: PARP-DBD expression in PSA-producing and PSA-negative cells. PSA-positive (LNCaP), PSA-insensitive (PC-3) prostate cell lines, and non-prostate (Ewing's sarcoma, A4573 cell line) cells were transiently transfected with pPSA(EP)-DBD/F or pCMV-DBD/F. Cells were harvested 48 hours after transfection and PARP-DBD expression was immunodetected as described in “Materials and Methods”.

FIG. 5: LNCaP cells were stably transfected with plasmid vectors that allow constitutive (pCMV-DBD/F) or androgen-inducible (pPSA-DBD/F) expression of PARP-DBD. The established cell sublines were analyzed for androgen-dependent induction of the PARP-DBD expression by Western blotting and RT-PCR. A, Immunodetection of PARP-DBD-Flag fusion protein in LNCaP cell sublines expressing PARP-DBD under control of CMV promoter (CMV-DBD) or PSA enhancer/promoter (PSA-DBD). LNCaP sublines stably transfected with pPSA (e/p)DBD/F were maintained in absence or in presence of synthetic androgen, R1881 (0-10 nM) as described in “Materials and Methods”. Parental LNCaP cells and LNCaP (CMV-DBD) cell subline were used as a negative and positive controls, respectively, for PARP-DBD expression. The migration of the DBD-Flag fused protein is indicated on the right. B, RT-PCR analysis of mRNA for PARP-DBD-Flag fused protein. LNCaP cells were stably transfected with vectors that allow constitutive (pCMV-DBD) or androgen-inducible (pPSA-DBD) expression of PARP-DBD. Specific RT-PCR product is indicated on the right, and molecular weight markers (M) are shown on the left.

FIG. 6: pPSA-DBD/F drives androgen-responsive expression of PARP-DBD in LNCaP cells. For in situ PARP-DBD immunodetection, LNCaP cells were grown in media containing 10% charcoal-stripped fetal bovine serum for seven days. Following induction of PARP-DBD expression by synthetic androgen R1881 (10 nM) for 24 h, cells were immunostained for PARP-DBD-Flag fusion protein as described in “Materials and Methods”. Transmitted (phase contrast) and Cy5 (red fluorescence) images were acquired using IX 70 confocal laser scanning microscope (Olympus). The same fields are shown. X200.

FIG. 7: PARP-DBD expression enhances DNA damage-induced growth inhibition in prostate carcinoma (LNCaP) cells. LNCaP cells were maintained in media containing 10% charcoal-stripped fetal bovine serum in presence (black columns) or absence (open columns) of synthetic androgen R1881 prior to irradiation (20 Gy) or treatment with etoposide (10 i). Viable cells were measured by an MTS assay at indicated times and results are expressed as a percentage of mock-treated control (n=4). Standard deviations are indicated.

FIG. 8: PARP-DBD sensitizes human prostate cancer cells to ionizing radiation and etoposide. LNCaP-DBD cells were maintained in absence or in presence of R1881 (10 nM) for 24 h prior to irradiation (20 Gy) or treatment with etoposide (10 μM). A, effect of PARP-DBD expression on annexin staining in LNCaP cells following to DNA-damaging treatments. Annexin V binding activity was determined by Flow cytometry 24 h after treatments. Apoptotic cells are defined as Annexin V positive cells and is expressed as percentage of total cell number in sample analyzed on FACS scan flow cytometer. Data presented are the mean values determined from triplicate experiments. B, effect of PARP-DBD expression on changes of mitochondrial membrane potential in LNCaP cells following to DNA-damaging treatments. 24 h after treatments, untreated controls (UT), irradiated (IR) or etoposide-treated (VP-16) cells were stained with JC-1 “DePsipher” reagent and analyzed by flow cytometry. Mitochondrial potential breakdown in dying cells results in accumulation of green fluorescent JC-1 monomers, which, in turn, is reflected by an increase of green fluorescence events.

### DETAILED DESCRIPTION OF A PREFERRED EMBODIMENT OF THE INVENTION

The following description will enable a person skilled in the art to which this invention pertains to make and use the invention, and sets forth the best modes contemplated by the inventors for carrying out the invention.

Prior to describing the preferred embodiments, the following definitions are provided.

Constructs or Compositions of the Invention

The tissue-specific constructs or compositions of the invention are constructs or compositions, which contain DNA encoding an inhibitor of DNA damage, repair under the control of tissue-specific transcriptional regulatory elements such as promoters and enhancers. Preferably, such constructs or compositions will comprise DNA encoding the DNA binding domain of poly(ADP-ribose)polymerase linked to the prostate specific antigen promoter and/or enhancer.

Apoptosis—

Programmed Cell Death.

Host—

A host includes humans, non-human primates, non-human mammals, and ungulates. Especially included are agricultural animals and domestic animals, such as dogs and cats.

The present invention is broadly directed to a method of treating diseases such as cancer comprising administering to a host a combination therapy that utilizes tissue-specific and treatment-specific therapies. The present invention further relates to the use of a DNA binding protein for the killing of specifically targeted cells in the treatment of cancer.

More specifically, the present invention provides a method for treating cancer by sensitizing cancer cells to genotoxic agents comprising administering to a host a therapeutically effective amount of a gene therapy composition comprising a construct encoding the DNA binding domain of poly(ADP-ribose) polymerase (PARP) and killing the targeted cancer cells by treating the host using conventional chemotherapeutic and/or radiation methods.

Although the preferred embodiments of the present invention as described below are in the context of prostate cancer, one skilled in the art will recognize that other cancers, e.g., brain cancer, stomach cancer, breast cancer, ovarian cancer, cervical cancer, prostate cancer, skin cancer, lung cancer, pancreatic cancer, liver cancer, colon cancer and leukemia, can be treated using the present invention without altering the scope of the invention.

In the search for methods to treat cancer, in particular prostate cancer, the inventors have observed the surprising and unexpected phenomenon that the DNA binding domain of (PARP) irreversibly binds to the ends of damaged DNA fragments. Due to this feature, PARP-DBD inevitably should mis-regulate DNA repair machinery, thus rendering cells sensitive to genotoxic treatments, such as chemotherapy or radiotherapy. The inventors have found that PARP-DBD sensitized LNCaP cells to DNA-damaging treatments such as ionizing radiation and etoposide (VP-16). Stimulation of PARP-DBD expression resulted in at least a two-fold higher rate of growth inhibition in prostate adenocarcinoma LNCaP cells compared to uninduced cells, and enhanced cell death of DBD-expressing cells in response to ionizing radiation (10-20 Gy) or etoposide (2,5-10 μM). Thus, exposure of PARP-DBD expressing prostate cancer cells to DNA-damaging treatments induces changes in the cell. These changes include but are not limited to impaired capacity for DNA damage repair, altered chromatin structure and reduced cells survival.

Experimental data indicate that a recombinant 28 kDa fragment of PARP containing the DNA binding domain (PARP-DBD) but lacking the automodification domain of PARP acts as a trans-dominant inhibitor of PARP activity by competing with intact PARP for DNA strand breaks. In previous studies the inventors have observed that the inhibition of PARP activity using chemical compounds, or depletion of endogenous PARP using antisense techniques resulted in impaired capacity for DNA damage repair, altered chromatin structure and reduced cell survival in response to exposure to DNA-damaging agents. Preliminary data indicate that PARP-DBD irreversibly binds to broken DNA strands making them inaccessible to DNA repair enzymes. As a result, cells containing unrepaired DNA damage are eliminated via activation of cell death pathways that may be executed by mechanisms of apoptosis or necrosis or by intermediate type (s) of cell death machinery.

Based on preliminary experimental results, some of which are discussed in the Examples below, it is hypothesized that the sustained presence of the DNA binding domain of PARP in prostate tissue kills tumor cells in response to massive DNA damage induced by ionizing radiation and/or genotoxic drugs.

The present invention is directed toward the use of the DNA binding domain of PARP to interfere with DNA repair machinery, thus sensitizing cells to genotoxic agents. Accordingly, the present invention will be used to treat diseases or conditions wherein genotoxic agents are used to kill cells. For example, the invention can be used to treat cancer, in particular prostate cancer, by sensitizing cancer cells to genotoxic agents such as radiation and/or chemotherapy.

Thus, it is a preferred embodiment of the invention to utilize the DNA binding domain of PARP to sensitize cells to genotoxic agents. Although the preferred embodiments are in the context of PARP-DBD as the agent to interfere with DNA repair, one skilled in the art will recognize that other proteins and compounds that mis-regulate or inhibit DNA repair can also be used without altering the scope of the invention.

The existence of tissue-specific promoters and enhancers, such as the PSA promoter and enhancer, provides the opportunity for targeting anti-cancer agents to the specific tissues. This can be done by introducing and selectively expressing a construct that encodes a potentially toxic protein, e.g., PARP-DBD, into specific cells. Thus, only cells of one particular type will be killed.

The 5′-regulatory sequences of the human PSA gene have been cloned (Riegman et al., Mol. Endocrinol. 5: 1921-1930 (1991)), and deletion analysis of this region identified a minimal (core) promoter region (−320 bp to +12), strong upstream enhancer (−5824 bp to −3738) and the presence of down-regulating elements within the central region (−4136 bp to −541) (Pang et al., Human Gene Therapy, 6: 1417-1426 (1995); Schuur et al., J. Biol. Chem., 271: 7043-7051 (1996); Pang et al., Cancer Res. 57: 495-499 (1997)). The 5′-enhancer linked to minimal core promoter has been shown to confer (i) prostate tissue specificity, (ii) androgen dependence, and (iii) enhanced gene expression (Schuur et al., J. Biol. Chem., 271: 7043-7051 (1996); Pang et al., Cancer Res. 57: 495-499 (1997)).

Thus, it is a preferred embodiment to use the transcriptional regulatory elements or sequences of the human PSA gene to drive the PARP-DBD expression in prostate cancer cells.

It is a more preferred embodiment to use the 5′-enhancer/core promoter of the PSA gene as an effective combination of regulatory sequences to drive the PARP-DBD expression in prostate cancer cells. The position of the enhancer within the PSA/PARP-DBD construct may be located at different positions from the promoter and may be in different orientations. A construct of the invention may contain multiple copies of the gene of interest to enhance production of the gene. The cassette of multiple enhancer elements upstream of minimal PSA promoter can also be used to improve the efficiency of the PARP-DBD expression in prostate cancer cells.

The preferred construct of the invention comprises the polynucleotide sequences of the toxic gene of interest, PARP-DBD, linked to the transcriptional regulatory sequences of the human PSA gene such that the PARP-DBD sequences are under the control of the PSA regulatory sequences.

Delivery of PSA/PARP-DBD: Gene Therapy

Gene therapy involves the introduction of one or more cloned genes of interest into cells. This may be achieved using viral or non-viral methods. Viral methods involve the use of viruses such as adenoviruses, retroviruses, herpes viruses, adeno-associated viruses and pox viruses.

Of the viruses mentioned, replication deficient Adenoviruses and adeno-associated viruses are typically used. Adenoviruses do not require cell proliferation to efficiently transfer genes to a cell and they are easily produced and purified. Thus, it is a preferred embodiment of the invention to use viral methods of gene therapy to introduce a construct or composition of the invention into target cells. It is a more preferred embodiment to use adeno-associated viruses or adenoviral vectors, such as Ad2 and Ad5.

The constructs or compositions of the invention can be introduced into cells via gene therapy methods such ex vivo or in vivo cell manipulation. Ex vivo gene therapy involves removing cells from a patient and introducing the gene of interest into the cells. The presence of the new gene in the cell is confirmed by methods well known in the art and the newly engineered cells are then returned to the patient.

A construct or composition of the invention can be introduced ex vivo by methods well known in the art, such as electroporation, transduction or transfection, into cells that have been removed from a patient.

In contrast, in vivo gene therapy involves administering to a host a construct or composition of the invention. A construct or composition of the invention can be introduced into cells using in vivo gene therapy methods known in the art such as liposomes or viruses.

For example, improved liposomal formulations could mediate significant level of gene expression in vivo. The liposomal formulation for efficient transfer of gene in vivo in mammalian cells is discussed in detail in U.S. Pat. No. 5,756,122 which is incorporated herein by reference.

A replication deficient retroviral vector can be used to introduce a construct of interest into cells. Methods involving the construction of retroviral expression vectors as well as methods for using these retroviral vectors in gene therapy are well in the art.

Retroviral vectors can be derived from viruses that include, but are not limited to, Rous Sarcoma Virus (RSV), Moloney Murine Leukemia Virus (MMLV), Human Immunodeficiency Virus (HIV) and Adenovirus.

In the event that viruses are used to transfer genes to target cells that are not the natural host of the virus, a modified virus comprising a binding moiety may be necessary. Binding moieties are heterologous proteins incorporated into the virus to allow the virus to bind to a target cell, which is not the natural target cell of the virus. Binding moieties include but are not limited to antibodies, fragments of antibodies, a molecule on the virus that is modified such that the binding specificity of the virus has changed, or a molecule added to the virus to change it binding profile. The production of viruses with binding moieties is discussed in detail in U.S. Pat. No. 5,885,808, which is incorporated herein by reference.

A construct of the invention may be administered to a human or other animal in an amount sufficient to produce a therapeutic, effect. Suitable construct of the invention, e.g., PSA/PARP-DBD or PSA/PARP-DBD packaged in an adenoviral vector, can be administered to such human or other animal in a conventional dosage form prepared by combining a construct or composition of the invention with a conventional pharmaceutically, cosmetically or dermatologically acceptable carrier or diluents according to known techniques. It will be recognized by one of skill in the art that the form and character of the pharmaceutically acceptable carrier or diluent is dictated by the amount of active ingredient with which it is to be combined, the route of administration and other well-known variables.

The route of administration of the construct or composition (e.g., PSA/PARP-DBD or PSA/PARP-DBD packaged in an adenoviral vector) of the invention may be oral, parenteral, by inhalation or topical. The term parenteral as used herein includes intravenous, intraperitoneal, intramuscular, subcutaneous, rectal or vaginal administration. Subcutaneous and intramuscular forms of parenteral administration are generally preferred. However, the preferred mode of administration will vary, e.g., dependent upon the particular condition treated.

The intermitted (weekly, monthly, or daily) parenteral and oral dosage regimens for administering the PARP-DBD construct according to the invention will generally be at concentrations that are achieved in humans under therapy for cancer. In a preferred embodiment, the daily parenteral and oral dosage regimens for employing compounds of the invention will be in the range of about 0.05 to 100, but preferably about 0.5 to 20, milligrams per kilogram body weight per day. The effective number of functional virus particles to be administered would range from 1×10⁵ to 5×10¹².

The construct or composition of the invention may also be administered by inhalation. By “inhalation” is meant intranasal and oral inhalation administration. Appropriate dosage forms for such administration, such as an aerosol formulation or a metered dose inhaler, may be prepared by conventional techniques. The preferred dosage amount of a compound of the invention to be employed is generally within the range of about 0.1 to 100 milligrams.

The construct or composition of the invention may also be administered topically. By topical administration is meant non-systemic administration and includes the application of a construct or composition of the invention externally to the epidermis, to the buccal cavity, an instillation of such a construct or composition into the ear, eye and nose, where it does not significantly enter the blood stream. This mode of administration is particularly desirable in the context of cosmetic or dermatological applications of the invention, e.g., in skin creams, ointments, or other topically administrable forms for the treatment of the skin. By systemic administration is meant oral, intravenous, intraperitoneal and intramuscular administration. The amount of a construct or composition required for therapeutic or prophylactic effect will, of course, vary with the nature and severity of the disease or condition being treated and the human or animal undergoing treatment, and is ultimately at the discretion of the physician. A suitable topical dose will generally be within the range of about 0.1 to 50 milligrams per kilogram body weight daily; or from 1×10⁵ to 5×10¹² functional virus particles per application.

The invention is also applicable to achieve the sensitization of other tumor cells (such as breast carcinoma, hepatoma etc) to DNA-damaging treatments when the PARP-DBD expression will be driven by promoters that specific to respective tissue.

DNA-Damaging Treatments

Treatments that cause DNA damage and have been used extensively include what are commonly known as γ-rays, X-rays, microwaves, electronic emissions, and/or the directed delivery of radioisotopes to tumor cells. It is most likely that these factors inflict a broad range of damages on DNA and affect DNA replication, gene expression and the assembly and maintenance of chromosomes. Dosage ranges for X-rays range from daily doses of 0.25 to 2.5 Gy for prolonged periods of time (3 to 4 weeks) to single doses of 20 to 60 Gy. Dosage ranges for radioisotopes vary widely, and depend on the half-life of the isotope, the strength and type of radiation emitted, and the uptake by the neoplastic cells.

Other agents that damage DNA include compounds also described as “Chemotherapeutic agents”. Agents such as cisplatin, and other DNA alkylating drugs may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally. Agents that damage DNA also include compounds that interfere with DNA replication, mitosis, and chromosomal segregation. Example of these compounds includes etoposide (VP-16), camptothecin and adriamycin, also known as doxorubicin, and the like. Widely used in clinical setting for the treatment of neoplasms these compounds are administered via injection intravenously at doses ranging from 25-75 mg/m² at 21 day intervals for adriamycin, to 35-50 mg/m² intravenously or double the intravenous dose orally.

The skilled artisan is directed to “Remington's Pharmaceutical Sciences” 15th Edition, chapter 33, in particular pages 624-652. Some variations in dosage will necessary occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biological Standards.

Materials and Methods

I. Cell Lines and Tissue Culture.

The androgen responsive prostate carcinoma LNCaP and androgen independent PC-3 cell lines were obtained from American Type Culture Collection (Manassas, Va.) and maintained by serial passage in DMEM supplemented with 10% fetal bovine serum, 100 units/ml penicillin, and 100 μg/ml streptomycin at 37° C. in an atmosphere of 5% CO₂ in air. Cells subjected to androgen stimulation tests were maintained in media with 10% charcoal-stripped fetal bovine serum for seven days before the addition of synthetic androgen R1881 (Perkin Elmer Life Science). Ewing's sarcoma cell line A4573 (kindly provided by Dr. T. Kinsella, University of Wisconsin, Madison) were maintained in Eagle's minimal essential medium (GIBCO) as described (Soldatenkov et al., Oncogene 18: 3954-3962 (1999)). All irradiations were performed at room temperature, in air, using a ¹³⁷Cs source in a “JL Shepard MARK I” laboratory irradiator at a dose rate of 3.85 Gy/min.

II. Recombinant DNAs.

DNA isolation, DNA sequencing, PCR amplifications and basic manipulations with recombinant DNAs were performed using protocols as described (Soldatenkov et al., Gene 161: 97-101 (1995); Jung et al., Science, 268:1619-1621 (1995); Soldatenkov et al., Oncogene 18: 3954-3962 (1999)).

RT-PCR Analysis.

RNA was isolated from cells using TRIzol Reagent (Gibco) according to manufacturer's protocol. The primers for human DBD-Flag fusion protein were: sense, 5′-ATCACCATCACCATCA-3′ and antisense, 5′-CCTTTATCGTCATCGT-3′. RT-PCR was performed using 2 μg of total cellular RNA and the ThermoScript RTPCR System (Gibco). The PCR amplification was carried out in a Perkin-Elmer amplification cycler (Wellesley, Mass.) during 35 cycles with denaturing at 960 C for 30 sec, annealing at 560 C for 30 sec, and extension at 720 C for 1 min. The amplified RT-PCR products were analyzed by 1% agarose gel electrophoresis, visualized by ethidium bromide staining and photographed under UV illumination.

Sample Preparation and Imaging with Atomic Force Microscopy

DNA samples or PARP-DNA binding reaction product in Mg²+containing buffer were deposited on an anatomically flat mica surface, allowed to adsorb for 1 min, rinsed with deionized water, and dried in a gentle nitrogen flow. The AFM images were obtained using a NanoScope IIIa instrument equipped with E-scanner (Digital Instruments, Santa Barbara, Calif.) operating in a tapping mode in air as described (Smulson et al., Cancer Res., 58: 3495-3498 (1998)). The tapping frequency of the 125 μm silicon cantilever was 300-400 KHz and the nominal scanning rate was set at 1-2 Hz. No less than 150 uncomplexed DNA molecules and 100 of PARP-DNA complexes were analyzed in each experiment.

DNA Transfections.

DNA transfections were carried out using an activated-dendrimer reagent (“Superfect”, Qiagen) as described (Soldatenkov et al., (1999) supra). Briefly, cells (2.0×10⁵) were plated into 60 mm tissue culture dishes coated with poly-L-lysine (Sigma) and maintained in culture for 2 days. Transfections were performed with 5 g of pCMV-DBD or pPSA-DBD plasmids using a ratio of DNA to “Superfect” reagent of 1:10. Cells were harvested 48 h after transfection for assays of PARP-DBD expression. Stable transfection of human prostatic adenocarcinoma cell line LNCaP and clonal selection were performed as previously described (Soldatenkov et al., (1995) supra; Soldatenkov et al., (1999) et al., supra).

VI. PARP-DBD Immunodetection.

Immunodetection of PARP-DBD Flag-fusion protein in human prostate carcinoma cells was performed as previously described (Soldatenkov et al., Cancer J Sci. Am., 3: 13-20 (1997)). In brief, logarithmically growing cells were washed twice with cold PBS and lysed at 4° C. for 30 min in buffer: 0.5% Triton X-100, 0.5% NP-40, 2 mM NaOV₄, 150 mM NaCl, 2 mM EDTA, 50 mM Tris-HCl (pH=7.5), 1 mM phenylmethylsulfonyl fluoride, 20 μg/ml aprotinin and 20 μg/ml leupeptin. Insoluble material was removed by centrifugation at 40 C for 30 min at 16,000×g and protein concentrations were determined using the “Micro BCA protein assay” (Pierce). Immunoprecipitation was performed by incubating the lysate with anti-Flag M2 monoclonal antibody agarose affinity gel (Sigma). Immune complexes were washed 6 times with 100 mM Tris (pH 7.5) -0.5% Tween buffer and subsequently resolved on NuPAGE Novex 4-12% gradient Bis-Tris gels (Invitrogen), followed by Western blotting using polyclonal anti-PARP antibody (R&D System; dilution 1:1,000) directed against the aa 71-329 of PARP protein. The secondary antibody was donkey anti-goat IgG (dilution 1: 2,000) conjugated to horseradish peroxidase (Santa Cruz). Signals were detected using the enhanced chemiluminescence system (Amersham). In some experiments, western immunoblot analyses for PARP were performed using rabbit polyclonal antibody (Cell Signaling, #9542) with a synthetic peptide (KLH coupled) corresponding to the caspase cleavage site in PARP (dilution 1:2000).

VII. Immunofluorescence and Phase Contrast Microscopy

For in situ PARP-DBD immunodetection, LNCaP cells were grown on poly-D-lysine-treated slides (Fisher Scientific) in media containing 10% charcoal-stripped fetal bovine serum for seven days. Following induction of PARP-DBD expression by synthetic androgen R1881 (10 nM) for 24 h, medium was removed and cells were subjected to fixation with 3.7% paraformaldehyde for 10 min as described (Soldatenkov and Dritschilo, Cancer Res, 57: 3881-3885 (1997); Soldatenkov et al., Cancer Res, 59: 5085-5088 (1999)). After rehydration in PBS, cells were permeabilized with PBS-0.2% Triton X-100 for 10 min, washed with PBS and incubated for 30 min with anti-Flag M2 monoclonal antibody (Sigma; dilution 1:200). Washes were followed by 30 min incubation with Cy-5 conjugated secondary antibody (Jackson ImmunoResearch, dilution 1:200) in PBS, contained 10% Donkey serum, 0.1% 300 Bloomgelatin. Slides were then washed with PBS, blotted dry and mounted with glass cover slips using a “Prolong Antifade” mounting medium (Molecular Probes, Inc). Confocal images (transmitted and Cy5 fluorescence) were acquired using IX 70 confocal laser scanning microscope (Olympus, Melville, N.Y.).

Proliferation Assay of Stably Transfected Cells

Growth characteristics of parental and stably transfected LNCaP cells were assayed by a colorimetric method using the tetrazolium compound, MTS (Cell Titer 96 Aqueous Assay; Promega) as we described previously (Soldatenkov et al., Oncogene 18: 3954-3962 (1999)). Briefly, exponentially growing cells were seeded in 96 well tissue culture plates at density of 2×1 03 cells per well and maintained for seven days in medium containing 10% charcoal-siripped fetal bovine serum. For induction of PARP-DBD expression, synthetic androgen R1881 (10 nM) was added to medium for 24 h prior to DNA-damaging treatments. Untreated cells (control), and cells treated with etoposide (10 μM) or irradiated (20 Gy) cells were incubated at 37° C. in humidified 5% C02 for up to fourteen days. The MTS reagent was added to the wells for 2 h and its bioreduction by cells was measured as absorbance at 490 nm using a 96 well plate reader (Bio-Tek Instruments, Inc). Each experiment was performed in quadruplicate and repeated at least three times.

Annexin V—Cell Death Assay.

Surface expression of phosphadtidyl serine was determined by Annexin V staining (Trevigen) according to the manufacturer's recommendations. Briefly, LNCaP cells (2-5×10⁵) were harvested, washed in PBS and exposed to FITC-labeled Annexin V. To exclude staining of phosphadtidyl serine on the inner surface of the cell membrane, indicative of cytoplasmic membrane disintegration, cells were counterstained with propidium iodite. FITC-Annexin V and propidium iodite staining was determined using a FACStar Plus flow cytometer (Becton Dickinson FACS System). Apoptotic cells were defined as FITC positive and PI negative.

Mitochondrial Depolarization Assay.

Changes in the mitochondrial potential were analyzed using the DePsipher kit (Trevigen) using JC-1 DePsipher reagent (5,5′, 6,6′-tetrachloro-1, 1′, 3,3′-tetraethylbenzimidazolylcarbocyanine iodite). This cationic dye forms red fluorescence aggregates in the mitochondria of healthy cells, and exists as a green fluorescence monomers following a collapse of mitochondrial potential in dying cells (Cossarizza et al., Biochem. Biophys. Res. Commun. 197: 40-45 (1993)). Briefly, cells were harvested, washed with PBS and exposed to JC-1 DePsipher reagent according to manufacturer's recommendations. Stained samples were analyzed at 488 nm argon laser by flow cytometry (FACStar Plus, Becton Dickinson FACS System).

The invention will now be described in more detail in the following Examples.

### EXAMPLE 1

Down Regulation of the PARP Expression Sensitizes Human Tumor Cells to Ionizing Radiation and DNA-Damaging Drugs

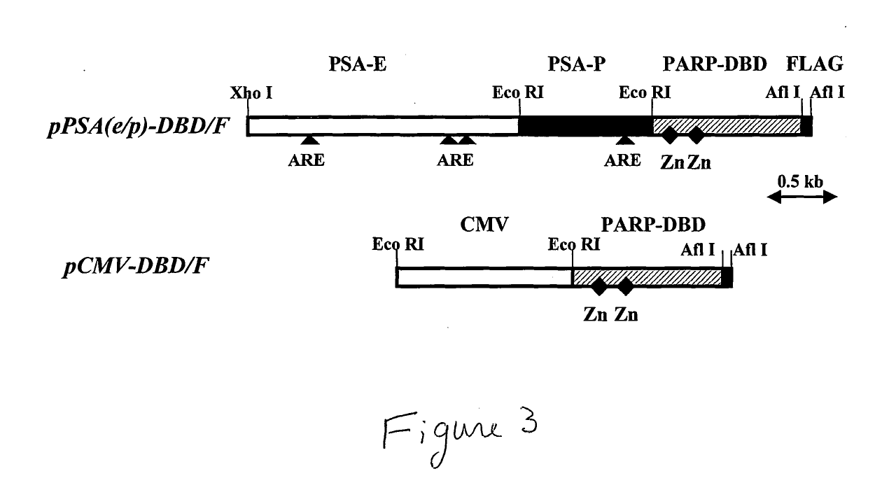

In preliminary studies applicants have established and characterized several mammalian cell lines stably transfected with antisense cDNA to PARP driven by an inducible promoter to dissect several accessory roles for this enzyme in a variety of DNA strand rejoining reactions (Stevnser, et al., Nucleic Acids Res., 22: 4620-4624 (1995); Ding et al., J. Biol. Chem., 267: 12804-12812 (1992); Ding et al., Cancer Res., 54: 4627-4634 (1994)). Antisense RNA transcripts were detected 5 hours after induction with dexamethasone, and remained relatively constant up to 48 hours in HeLa cells followed by partial degradation by 72 hours. Importantly, dexamethasone reduced the amount of PARP mRNA in antisense cells by approximately 5% of the original value but had no effect on PARP expression in control cells. Using this antisense-RNA-expression approach, authors have shown that the initial rate of DNA repair after exposure to methylmethanesulfonate (MMS) is significantly inhibited in PARP-depleted HeLa cells (Ding et al., J. Biol. Chem., 267: 12804-12812 (1992)). Furthermore, the data from our investigations have demonstrated that the depletion of endogenous PARP in cells followed by exposure to DNA-damaging agents resulted in reduced cell survival (FIG. 3) and altered chromatin structure (Ding et al., Cancer Res., 54: 4627-4634 (1994)).

### EXAMPLE 2

Expression and Purification of the DNA-Binding

### Domain of PARP (PARP-DBD)

In order to explore the significance of DNA-binding domain of PARP in programmed cell death, Applicants isolated the PARP cDNA fragment encompassing the region that encodes two zinc fingers of the enzyme as well as the KKKSKK nuclear localization signal. Clone pCD12, containing the full length cDNA encoding human PARP in an Okayama-Berg vector (Cherney et al., Proc. Natl. Acad Sci. USA, 84: 8370-8374 (1987), was used as a template for construction of a PARP-DBD expression vector. PCR was performed with a 28 bp primer that contained a Bam HI restriction site upstream (nt 164-180) of PARP c DNA, and a 22 bp primer that contained a Hind III restriction site downstream (nt 837-854) of PARP cDNA. The DBD of PARP was subsequently cloned into bacterial expression vector pQE30 (Qiagen), expressed and purified to 95% homogeneity by affinity chromatography as previously described (Rosenthal et al., Nucleic Acids Res., 25: 1437-1441 (1997)).

### EXAMPLE 3

Irreversible Binding of PARP-DBD to DNA Ends in Apoptotic Cells

Applicants have investigated the interactions of PARP and its fragment containing the DNA-binding domain (DBD) with DNA by atomic force microscopy, which reveals the topography of DNA and proteins at nanometer resolution.

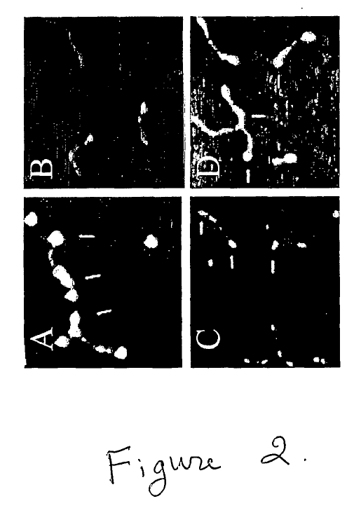

Experiments revealed that recombinant full length PARP bound plasmid DNA fragments and linked them into chainlike structures (FIG. 2A). Upon automodification of PARP in the presence of NAD⁺, PARP dissociated from the DNA fragments, which, nevertheless, remained physically aligned (FIG. 2B). A recombinant 28-kDa fragment of PARP containing the DNA-binding domain but lacking the automodification domain irreversibly bound to and linked DNA fragments in the absence (FIG. 2C) or presence (FIG. 2D) of NAD⁺.

Interestingly, identical results were obtained upon incubation of internucleosomal DNA fragments from apoptotic cells with products of cleavage of recombination PARP by purified caspase-3 (Smulson et al., Cancer Res., 58: 34953498 (1998)). These data are consistent with the proposed model for protection of DNA breaks from repair enzymes by PARP-DBD (Satoh et al., Nature (Lond.), 356: 356-358 (1992); Smulson et al., Biochemistry, 33: 6186-6191 (1994)).

### EXAMPLE 4

Cloning of the PSA Promoter Region and Construction of the PARP-DBD Expression Plasmids

The 5′-regulatory sequences of the human PSA gene have been cloned (Riegman et al., Mol. Endocrinol. 5: 1921-1930 (1991)). Deletion analysis of this region identified a minimal (core) promoter region (−320 bp to +12), strong upstream enhancer (−5824 bp to −3738) and the presence of down-regulating elements within the central region (−4136 bp to −541) (Pang et al., Human Gene Therapy, 6: 1417-1426 (1995); Schuur et al., J. Biol. Chem., 271: 7043-7051 (1996); Pang et al., Cancer Res. 57: 495-499 (1997)). The 5′-enhancer linked to minimal core promoter has been shown to confer (i) prostate tissue specificity, (ii) androgen dependence, and (iii) enhanced gene expression (Schuur et al., J. Biol. Chem., 271: 7043-7051 (1996); Pang et al., Cancer Res. 57: 495-499 (1997)). These features suggest that 5′-enhancer/core promoter is an effective combination of PSA gene regulatory sequences to drive the PARP-DBD expression in prostate cancer cells.

A PCR-generated probe (nts 1-200 of PSA cDNA) was used to screen a human placenta genomic library. Two identical clones were isolated and genomic fragments were further analyzed. The 1.3 kb fragment that contains the upstream enhancer element of the PSA regulatory region (nt −5030 to −3749) was identified by hybridization with the same probe used earlier and subcloned into pcDNA 3.1 (−) expression vector (Invitrogen). The PSA promoter region (nt −664 to +30) was amplified by PCR using human placenta genomic DNA as a template and the 20 bp primers: 5′-GGTCTGGAGAACAAGGAGTG (forward primer) and 5′TCTCCGGGTGCAGGTGGTAA (reverse primer). The resulting PCR product was directly cloned in pCRII vector (Invitrogen) and sequenced to verify its fidelity. The 1.1 kb EcoR I—Hind III fragment of the human PARP cDNA encoding for DBD was isolated as previously described (Rosenthal et al., 1997).

Following Recombinant Plasmids were Constructed:

(i) The human cDNA coding for the DNA-binding domain of PARP (5′-Eco RI—Hind III) was inserted into pcDNA 3.1 (−) expression vector (Invitrogen) at EcoRi/Hind III restriction sites downstream of the human cytomegalovirus (CMV) promoter/enhancer. Subsequently, PARP-DBD was tagged at its carboxy terminus with a sequence encoding four FLAG-epitope tags. The resulting recombinant plasmid, pCMV-DBD/F, permits constitutive expression of human PARP-DBD under control of the CMV promoter (FIG. 3).

(ii) The pCMV-DBD/F plasmid was modified to remove Nru I-Pme I fragment that contained the CMV promoter sequences giving rise to pΔCMV-DBD plasmid. This vector is used as a control in transient and stable transfections.

(iii) Next, two basic vectors for expression of the human PARP-DBD under control of the PSA gene regulatory elements were generated. An Eco RI fragment (nt −633 to +30) containing 662 bp sequence of PSA promoter was cloned into Eco RI site of pΔCMV-DBD giving rise to pPSA (P)-DBD/F. To generate a pPSA (EP)-DBD/F plasmid, a 1281 bp Xho I—Bam HI fragment of PSA enhancer was modified to generate a blunt end at Bam HI site and subsequently was inserted upstream of PSA promoter into pPSA (P)-DBD/F at Xho I/Eco RV restriction sites.

The resulting plasmid, pPSA (EP)-DBD/F (FIG. 3), permits the expression of the human PARP-DBD in androgen-inducible and PSA-dependent fashion. Resulting recombinant plasmids were analyzed with restriction enzymes, and sequences are confirmed to be in-frame.

### EXAMPLE 5

PARP-DBD Expression in PSA-Producing and PSA-Negative Human Tumor Cells

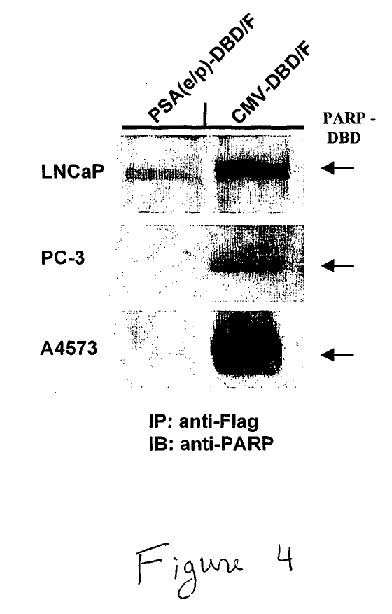

To determine whether PARP-DBD can be expressed in mammalian cells, prostate carcinoma cell lines LNCaP and PC-3 and Ewing's sarcoma (A4573 cell line) cells were transiently transfected with the pCMV-DBD/F plasmid that allow constitutive expression of PARP-DBD. DNA transfections were carried out using an activated-dendrimer reagent (“Superfect”, Qiagen) essentially as we described (Soldatenkov et al., Oncogene 18: 3954-3964 (1999)). Applicants demonstrated that expression of PARP-DBD-Flag fusion proteins can be reliably detected by Western immunoblotting in all tested cell lines for at least 48 hours post-transfection (FIG. 4).

Tissue specificity of PARP-DBD expression under control of PSA promoter/enhancer was evaluated in transient transfection assays using the PSA-positive (LNCaP) and PSA-negative (PC-3) prostate cancer cells and cells of nonWO prostate origin such as Ewing's sarcoma (A4573 cell line). Applicants found that PSA enhancer/promoter driven expression of the human PARP-DBD was observed only in PSA-producing LNCaP prostate carcinoma cells but not in PSA-independent cell lines, at least at immunodetectable levels (FIG. 4).

### EXAMPLE 6

Establishment of Stable Transfected LNCaP Cell Lines.

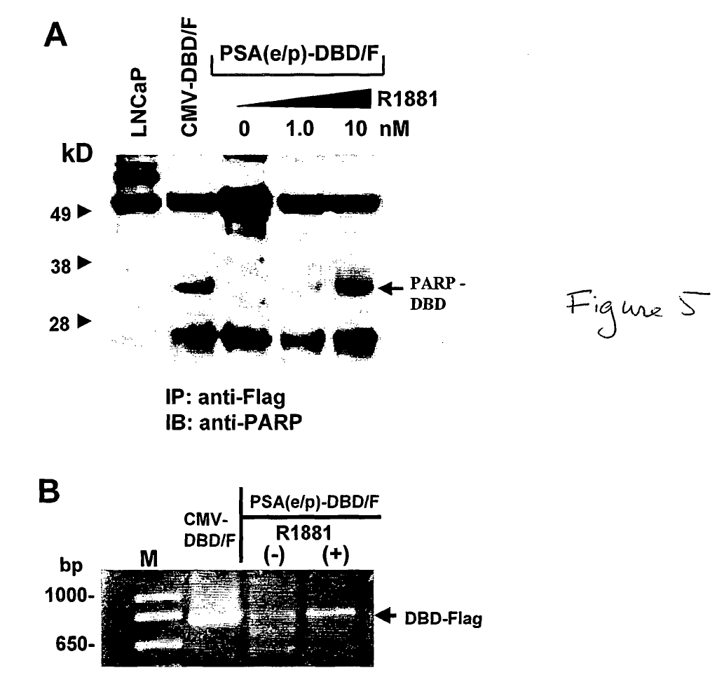

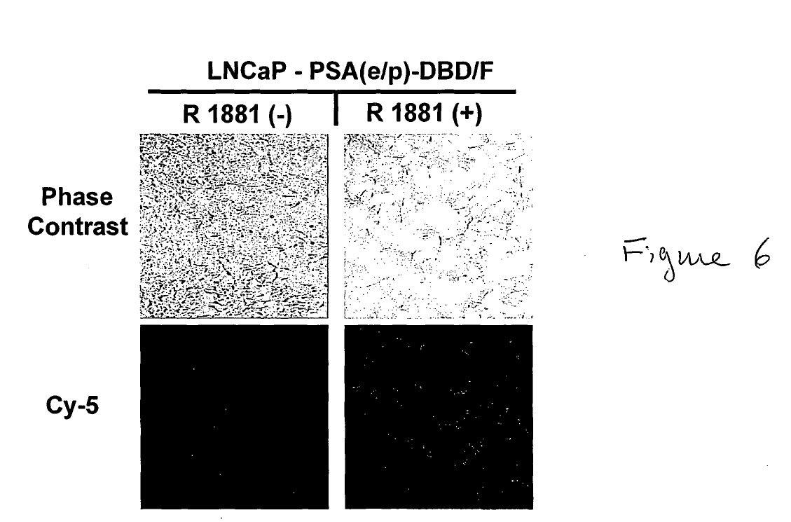

LNCaP cells were transfected with pPSA (e/p) -DBD, pCMV-DBD or with control, neomycin-resistant expression vector pΔCMV-DBD, respectively, using an activated-dendrimer reagent (“Superfect”, Qiagen). Transfection medium was replaced with complete growth medium and cells were incubated for 48 h to allow expression of neomycin-resistance, followed by replating into selective medium containing 300 μg/ml G418 (Geneticin; GIBCO). The G418-resistant colonies from each replicated experiment were pooled to form polyclonal cell populations and were routinely maintained in medium containing 300 μg/ml G418. Established polyclonal LNCaP sublines were subsequently subjected to screening for androgen-dependent PARP-DBD expression (see below, FIG. 5 and FIG. 6).

### EXAMPLE 7

Androgen-Regulated Expression of PARP-DBD Under Control of PSA Promoter/Enhancer in LNCap Cells.

LNCaP cells stably transfected with pPSA (e/p)-DBD/F (see example 6 for description) were grown in media containing charcoal-stripped serum for seven days followed by incubation for 24 hours in absence or in presence of synthetic androgen, R1881 (0-10 nM). Androgen-regulated expression of the human PARP-DBD in LNCaP cells was assessed by Western blot analysis (FIG. 5A), RT-PCR (FIG. 5B) and in situ immunofluorescence (FIG. 6). Parental LNCaP cells and LNCaP cell subline (CMV-DBD) were used as a negative and positive controls, respectively, for PARP-DBD expression. Applicants found that exposure of LNCaP cells stably transfected with pPSA (e/p)-DBD/F to androgen (R1881) resulted in dose-dependent stimulation of PARP-DBD expression at levels of mRNA and protein (FIGS. 5A and 5B). Androgen-regulated expression of human PARP-DBD in these cells was further confirmed by in situ immunodetection of DBD-Flag fusion protein using fluorescence microscopy (FIG. 6).

### EXAMPLE 8

The Sensitization of Prostate Cancer Cells to Ionizing Radiation and DNA-Damaging Drugs by PARP-DBD

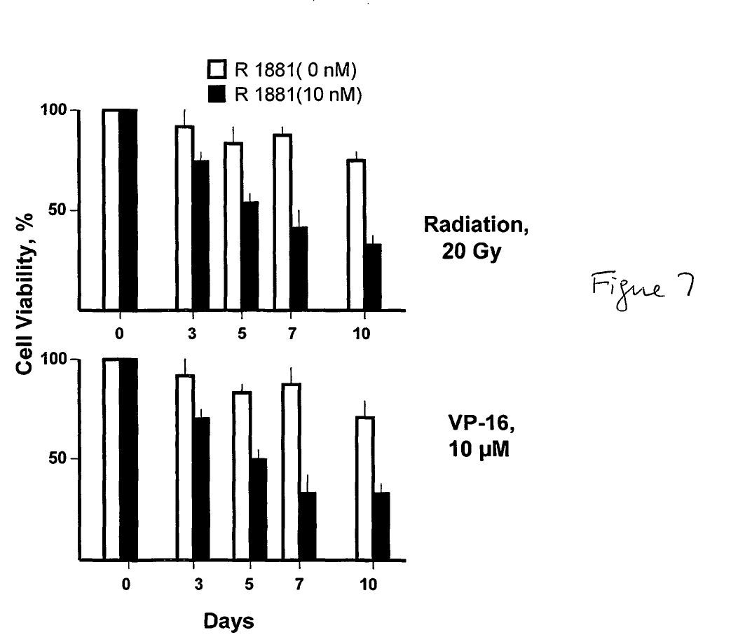

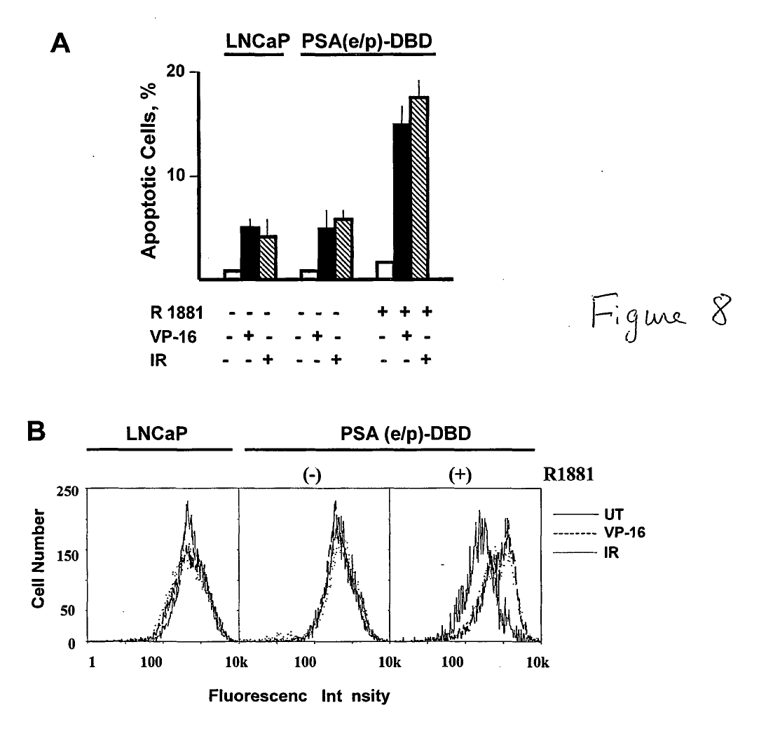

Applicants found that PARP-DBD expression sensitized LNCaP cells to DNA-damaging treatments such as ionizing radiation and etoposide (VP-16). Androgen (R1881)-dependent stimulation of PARP-DBD expression resulted in at least a two-fold higher rate of growth inhibition in LNCaP cells compared to uninduced cells in response to ionizing radiation or etoposide (FIG. 7). This effect was observed beginning 72 hours post treatment and appeared to be strictly related to the level of expression of PARP-DBD (FIG. 5). Data from literature (Kimura et al., Cancer Res. 59: 1606-1614 (1999)), and our studies (FIG. 7 and FIG. 8) show that LNCaP cells are highly resistant to radiation-induced cell death. Both parental LNCaP cells and un-induced LNCaP-DBD cell sublines showed only modest increase of annexin V-binding activity (indicative of early apoptotic changes) in response to ionizing radiation or etoposide (FIG. 8). In contrast, when PARP-DBD expression was induced by R1881, irradiated or etoposide-treated LNCaP cells showed significantly (more than two fold) increased staining for Annexin V and depolarization of mitochondrial membrane by 24 hours post treatment (FIG. 8).

These data indicate that inhibition of PARP function by means of enforced expression of its dominant negative mutant (PARP-DBD) results in sensitization of human prostate cancer cells to DNA damaging treatments.

To conclude, the plasmid vector developed in this study permits the expression of the human PARP-DBD in an androgen-inducible and PSA-dependent fashion and sensitizes prostatic adenocarcinoma cells to DNA-damaging treatments. These results provide a proof-of-principle for a novel therapeutic strategy to control prostate cancer.

What is claimed is:

1. A recombinant DNA construct comprising the coding region of the DNA binding domain of poly(ADP-ribose) polymerase linked to tissue-specific transcriptional regulatory sequences.

2. The recombinant DNA construct of claim 1, wherein said tissue specific transcriptional regulatory sequences are selected from the group consisting of promoters and enhancers.

3. The DNA construct of claim 2, wherein said promoter is the prostate specific antigen promoter.

4. The DNA construct of claim 2, wherein said enhancer is the prostate specific antigen enhancer.

5. An expression vector comprising the recombinant DNA construct of claim 1.

6. A method of treating cancer comprising: (1) sensitizing cancer cells by administering to a host a therapeutically effective amount of a construct of claim 1, and (2) inducing apoptosis of cancer cells by treating the host by genotoxic treatments.

7. The method of claim 6, wherein said administration is oral, parenteral, inhalation, topical, or by gene therapy.

8. The method of claim 6, wherein said host is a human diagnosed with cancer.

9. The method of claim 6, wherein said cancer is selected from the group consisting of brain cancer, stomach cancer, breast cancer, ovarian cancer, cervical cancer, prostate cancer, skin cancer, lung cancer, pancreatic cancer, liver cancer, colon cancer and leukemia.

10. The method of claim 6, wherein said genotoxic treatments comprise chemotherapeutic drugs or radiation.

11. The method of claim 10, wherein said radiation and waves that induce DNA damage are as γ-irradiation, X-rays, microwaves, electronic emissions, and the like.

12. The method of claim 10, wherein said chemotherapeutic drugs are alkylating agents, inhibitors of DNA replication, mitosis, or chromosomal segregation, and radiomimetic agents.

13. The method of claim 12, wherein said alkylating agents are cis-diamine dichloroplatinum or melphalan.

14. The method of claim 12, wherein said inhibitors are etoposide (VP-16), camptothecin and adriamycin, also known as doxorubicin.

15. The method of claim 12, wherein said radiomimetic agent is bleomycin.

16. A method of treating cancer comprising: (1) sensitizing cancer cells by administering to a host a therapeutically effective amount of a recombinant DNA construct comprising the coding region of a DNA repair inhibitory agent linked to tissue-specific transcriptional regulatory sequences and (2) inducing apoptosis of cancer cells by treating the host by genotoxic treatments.

17. The method of claim 16, wherein said inhibitory agent inhibits the function of a protein selected from the group consisting of c-jun, c-fos, poly-ADP ribose polymerase, DNA polymerase .beta., topoisomerase I, d-TMP synthase, hMTII-A, uracil DNA glycosylase, alkyl-N-purine DNA glycosylase, DNA ligase IV, DNA ligase III, Hap-1, Ref-1, poly-ADP ribose polymerase and DNA-dependent protein kinase.

18. The method of claim 16, wherein said inhibitory agent is the DNA binding domain of poly(ADP-ribose) polymerase.

19. The method of claim 16, wherein said tissue specific transcriptional regulatory sequences are selected from the group consisting of promoters and enhancers.

20. The method of claim 19, wherein said promoter is the prostate specific antigen promoter.

21. The method of claim 19, wherein said enhancer is the prostate specific antigen enhancer.

22. The method of claim 16, wherein said cancer is selected from the group consisting of brain cancer, stomach cancer, breast cancer, ovarian cancer, cervical cancer, prostate cancer, skin cancer, lung cancer, pancreatic cancer, liver cancer, colon cancer and leukemia.

23. A method for the induction of apoptosis in a cell comprising introducing into said cell a recombinant DNA construct comprising the coding region of a DNA repair inhibitory agent linked to tissue-specific transcriptional regulatory sequences.

24. The method of claim 23, wherein said inhibitory agent inhibits the function of a protein selected from the group consisting of c-jun, c-fos, poly-ADP ribose polymerase, DNA polymerase beta., topoisomerase I, d-TMP synthase, hMTII-A, uracil DNA glycosylase, alkyl-N-purine DNA glycosylase, DNA ligase IV, DNA ligase III, Hap1, Ref-1, poly-ADP ribose polymerase and DNA-dependent protein kinase.

25. The method of claim 31, further comprising the step of providing to said cell a DNA-damaging agent.

26. The method of claim 25, wherein said DNA-damaging agent is selected from the group consisting of cisplatin, carboplatin, VP16, teniposide, daunorubicin, doxorubicin, dactinomycin, mitomycin, plicamycin, bleomycin, procarbazine, nitrosourea, cyclophosphamide, bisulfan, melphalan, chlorambucil, ifosfamide, merchlorehtamine, taxol, taxotere, anthracyclines and ionizing radiation.

27. The method of claim 23, wherein said cell is a tumor cell.

28. The method of claim 27, wherein said tumor cell is selected from the group consisting of lung tumor cell, a prostate tumor cell, a breast tumor cell, a colon tumor cell, a liver tumor cell, a brain tumor cell, a kidney tumor cell, a skin tumor cell and an ovarian tumor cell.

29. The method claim 27, wherein said tumor cell is selected from the group consisting of a squamous cell carcinoma, a non-squamous cell carcinoma, a glioblastoma, a sarcoma, a melanoma, a papilloma, a neuroblastoma and a leukemia cell.

30. The construct of claim 1 comprising the recombinant plasmid pCMV-DBD/F.

31. The method of claim 6, wherein the construct comprises the recombinant plasmid pCMV-DBD/F.

32. The method of claim 16, wherein the construct comprises the recombinant plasmid pCMV-DBD/F.

33. The method of claim 23, wherein the construct comprises the recombinant plasmid pCMV-DBD/F.

34. The construct of claim 1 comprising the recombinant plasmid pPSA(e/p)-DBD/F.

35. The method of claim 6, wherein the construct comprises the recombinant plasmid pPSA(e/p)-DBD/F.

36. The method of claim 16, wherein the construct comprises the recombinant plasmid pPSA(e/p)-DBD/F.

37. The method of claim 23, wherein the construct comprises the recombinant plasmid pPSA(e/p)-DBD/F.


SEQUENCE LISTING (5 sequences)

SEQ ID NO: 1 - DNA, 16 bp, Artificial Sequence
  Description of Artificial Sequence Primer
atcaccatcaccatca

SEQ ID NO: 2 - DNA, 16 bp, Artificial Sequence
  Description of Artificial Sequence Primer
cctttatcgtcatcgt

SEQ ID NO: 3 - protein, 6 aa, Homo sapiens
  nuclear localization signal
KKKSKK

SEQ ID NO: 4 - DNA, 20 bp, Artificial Sequence
  Description of Artificial Sequence Primer
ggtctggagaacaaggagtg

SEQ ID NO: 5 - DNA, 20 bp, Artificial Sequence
  Description of Artificial Sequence Primer
tctccgggtgcaggtggtaa